(19) United States
(12) Patent Application Publication
(10) Pub. No.: US 2020/0306424 A1
(43) Pub. Date: Oct. 1, 2020

(54) ASPIRATOR

(71) Applicant: STEVEN SUNG-KYUN JUHN, ST. PAUL, MN (US)

(72) Inventor: STEVEN SUNG-KYUN JUHN, ST. PAUL, MN (US)

(21) Appl. No.: 14/835,017

(22) Filed: Aug. 25, 2015

Related U.S. Application Data
(63) Continuation of application No. 62/070,393, filed on Aug. 25, 2014.

(51) Int. Cl. A61M 1/00

(52) U.S. Cl. CPC A61M 1/0031

(57) ABSTRACT

An aspirator for manually collecting liquid sample, particularly for medical purposes, comprising a handle connected to a vacuum source and having a sample collection vial fitting into a cavity in the handle with a thumb control vent slot in the top of the handle to selectively control suction required to collect the liquid sample. A tubing functions as the probe for the sample to be collected. The tubing leads into the sample collection vial.

### CROSS REFERENCE TO RELATED APPLICATION

This application claims the priority of U.S. Application Ser. No. 62/070,393 filed Aug. 25, 2014.

### FIELD OF THE INVENTION

The invention is in the art of liquid collecting devices, particularly collection devices having aspirators for fluid samples from body cavities.

### BACKGROUND OF THE INVENTION

There are occasions when it is desirable or necessary to obtain a small specimen of a liquid from a location which is difficult to reach. Important examples of such instances are found in the diagnosis of various diseases, infections and other ailments of the human body. While instruments of various types have been employed in the past to collect samples for medical analysis they have, in general, been of a permanent type requiring sterilization after each use. The modern tendency is to employ instruments which may be used once and discarded so as to avoid the high cost of assembly, sterilization, and repackaging to maintain the sterile condition. Also previous instruments have had difficulty in collecting samples of various viscosity. Otitis media is a middle ear infection which is extremely common and manifests itself with the presence of fluid in the middle ear. This fluid may be serous, purulent, or mucoid having different viscosity and may arise as a result of several different circumstances or etiologies. In order to properly treat the patient it is important to collect a sample of the fluid and analyze it.

### SUMMARY OF THE INVENTION

It is an object of the present invention to provide a nonreusable aspirator designed to collect small specimens of liquid samples, particularly from the human body. It is a particular object of this invention to provide such an instrument for use in collecting liquid samples from the middle ear using variable suction. The present invention involves a simple, inexpensive apparatus which can be employed to collect such fluid into a sample vial which can then be sent for suitable analysis and the entire apparatus thrown away.

This invention contemplates an aspirator for manually collecting samples of fluids from remote cavities comprising an elongated handle with a top, a bottom, a forward end, a rearward end, a centrally located cavity on the bottom of the handle, a passageway communicating the cavity with both the forward and rearward ends, and an aspiration controlled finger vent communicating the cavity with the top, a removable specimen collection vial having an open top attaching to said cavity with an airtight fitting, a removable flexible tube extending from said cavity through said passageway and out and beyond said forward end for a selected distance, said tube providing an airtight seal with said passageway, and said rearward end being adapted for connection of said passageway to a source of vacuum which is selectively controlled with the vent. The aspirator is designed for collection of liquids of varying viscosity from the middle ear of a human by employing either a flexible plastic tube or a Luer-Lok hypodermic needle to reach into the middle ear cavity through the tympanic membrane.

### DESCRIPTION OF THE DRAWING

FIG. 1 is a perspective view of the aspirator for collecting liquid samples of the invention connected to a vacuum source;

FIG. 2 is an enlarged top plan view thereof;

FIG. 3 is a sectional view taken along line 3-3 of FIG. 2;

FIG. 4 is a sectional view taken along line 4-4 of FIG. 2;

FIG. 5 is a sectional view taken along line 5-5 of FIG. 2;

FIG. 6 is a sectional view taken along line 6-6 of FIG. 2;

FIG. 7 is a sectional view of the air control portion of the aspirator with a thumb to control flow of air into the aspirator;

FIG. 8 is a sectional view similar to FIG. 7 showing a thumb closing about one-half of the air opening; and

FIG. 9 is a sectional view similar to FIG. 7 showing a thumb closing about three-quarters of the air opening.

### DESCRIPTION OF PREFERRED EMBODIMENT

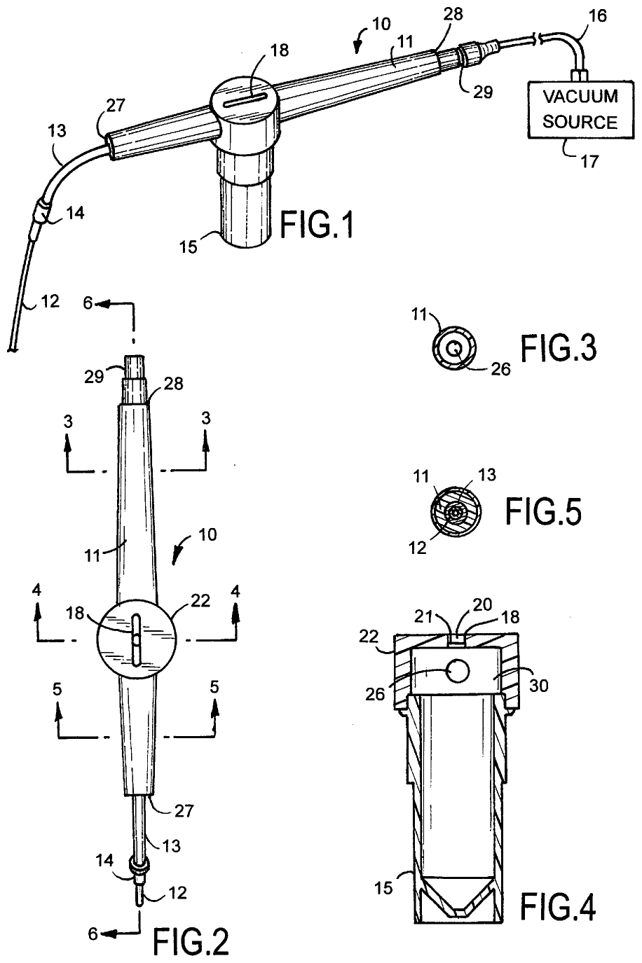

The general features of this invention can best be understood with respect to FIG. 1. The apparatus comprises a generally elongated tubular shaped handle 11 which has an internal passageway leading from its forward end 27 and from its rearward end 28 to a central cavity 30 which is on the bottom of handle 11 and which fits collection vial 15. The passageway at the rearward end 28 of handle 11 is connected to a source of vacuum 17 by a flexible tube 16. The passageway at forward end 27 of the handle 11 is connected to a flexible tube 12 which extends from sample vial 15 to some selected distance beyond forward end 27 which is necessary and desirable to reach the cavity from which the liquid specimen is to be collected. Vial 15 is made with a neck at its open end and a projecting collar which permits vial 15 to be inserted into a corresponding cavity in the bottom of handle 11 to produce a connection which is substantially airtight. A soft metal tube 13 fits into the passageway in handle 11. Through the center of tube 13 is threaded a soft flexible plastic tube 12. The relative dimensions of metal tube 13 and plastic tube 12 are such that they provide an airtight seal with respect to the passageway in handle 11 at forward end 27. There is also shown in this drawing cuff 14 which is a short length of soft tubing fitting over the free end of metal tube 13 and the forward extension of plastic tube 12. Cuff 14 is preferably made of a silicone rubber. Cuff 14 can be made of other materials which are soft and pliable.

On a center portion of the upper surface of handle 11 there is a generally cylindrical shaped raised member 22 having an aspiration control thumb vent 18 which communicates with cavity 30 in the bottom of handle 11 into which sample vial 15 fits. Vent 18 is a generally linear slot in the upper surface of raised member 22 which extends generally parallel to passageway 26. Vent 18 has downwardly inclined bottom surfaces 19 and 20 and a centrally located opening 21 which opens to cavity 30. Opening 21 is located below the upper surface of raised member 22. Vent 18 permits the operator of the device to apply suction through tube 12 when the operator's thumb 32 closes vent 18 to the atmosphere. When vent 18 is left uncovered or is partially uncovered, however, the vacuum is broken or partially broken with respect to tube 12 since vacuum from source 17 draws air from atmosphere through vent 18 rather than through the more difficult path of tube 12. Incremental adjustment of the suction is easily accomplished by sliding thumb 32 of the operator on the upper surface of raised member 22 to progressively close or open vent 18 as necessary to regulate vacuum pressure in tube 12 so as to collect the fluid specimen depending on the viscosity of the fluid. Additionally, source 17 can be operatively connected to a regulator to regulate the vacuum pressure applied to the apparatus to facilitate collection of fluid of varying viscosity from remote body cavities.

In some instances the fluid which is collected as a specimen may be of a thick viscous nature which will not readily flow through the internal diameter of tube 12. If the liquid is sufficiently fluid to fill a portion of tube 12, the liquid may be expelled from the tube in vial 15 by applying pressure to the tube or by squeezing between tweezers or pliers to force the fluid out of the tubing. If the collective liquid is even more viscous so that it approaches a semisolid condition it is entirely feasible to merely cut the filled tubing into short lengths and submit the specimen in that form for analysis.

In some instances, the surgeon may wish to employ a hypodermic needle to puncture the tympanic membrane and to remove the sample. For such purposes the Leur-Lok needle can be employed. This type of needle has a socket and it is for purposes of seating this socket that cuff 14 is employed. In this instance tube 12 is severed near the end of cuff 14. The Leur-Lok needle can then be manually seated over cuff 14 to produce a rigid airtight seal.

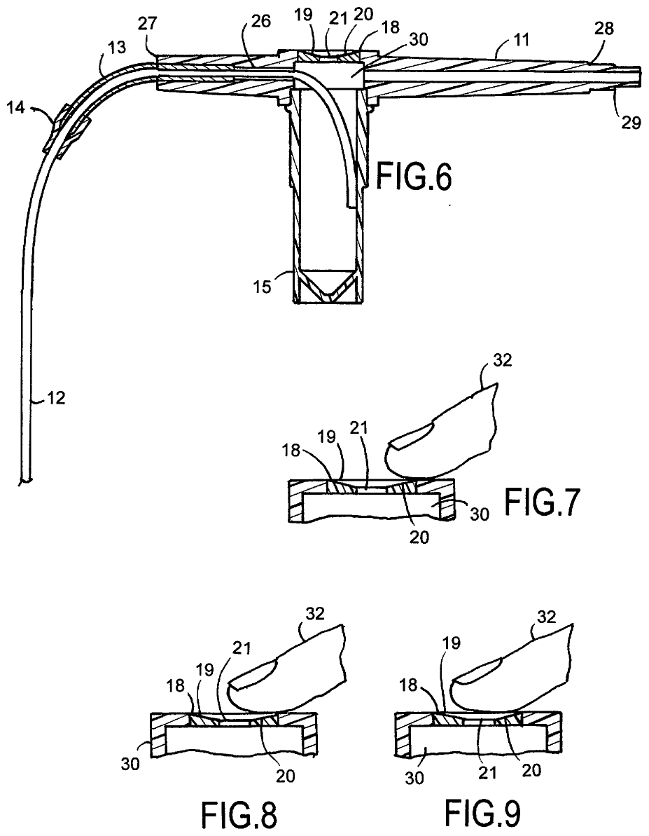

In FIG. 6 the details of construction of the device of this invention can be readily understood. Handle 11 contains a central cavity 30 and a passageway 26 which extends from the forward end 27 to cavity 30 and from the rearward end 28 to cavity 30. At rearward end 28 there is shown a typical fitting 29 designed for the attachment of flexible tubing 16 to produce an airtight fitting so that vacuum may be selectively controlled with vent 18 and drawn through the hollow portion of tubing 16 and passageway 26. Inserted into passageway 26 in forward end 27 is a soft metal tube 13 which typically may be aluminum. Through the hole in soft metal tube 13 is inserted a soft flexible tubing 12 extending from vial 15 in cavity 30 through tube 13 and beyond to whatever selected distance is preferred. The dimensions of flexible tube 12, metal tube 13 and passageway 26 are such that a substantially airtight fitting is obtained by manually assembling tubes 12 and 13 into passageway 26. Tube 12 is preferably made of a polyamide. Handle 11 is preferably made of an injection moldable grade of polyamide. Tube 12 terminates near the bottom of vial 15 whereby any vacuum applied through passageway 26 will be transmitted through tube 12 if vent 18 is partially closed or fully closed. Flexible tubing 16 may be made of rubber, polyamide, or the like.

This device is eminently suitable for use with a myringotomy incision when tube 12 is employed to collect fluid from the middle ear. If the surgeon prefers to employ a hypodermic needle, e.g., for purposes of tympanocentesis, the needle may be employed to puncture the tympanic membrane and the sample collected. It is of course entirely possible that the device of this invention can be employed for collecting fluid samples from other locations whether they be from human cavities that are difficult to reach or from cavities which are difficult to reach and wherein a small sample, e.g. 1-2 cc, is to be collected. Such might be the case in a variety of laboratory techniques, for dental purposes, for veterinarian purposes, or the like.

There has been shown and described embodiments of the aspirator of the invention. Changes in the materials, structures, markings, and arrangement of structures may be made by persons skilled in the art without departing from the invention.

1. An aspirator for manually collecting samples of fluids from remote human body cavities comprising:
an elongated axially extending handle having a top surface, a bottom surface, a forward end, a rearward end and a centrally located cavity opening through the bottom surface of the handle,
the handle between the cavity and the rearward end adapted to be manually gripped,
a rigid longitudinal passageway extending through the handle communicating the cavity with the forward end and the rearward end of the handle,
a cylindrical raised member extending upwardly from a central portion of the top surface of the handle and enclosing a top portion of the cavity,
the raised member having a flat circular upper surface, the upper surface of the raised member having an aspiration control vent located above the top portion of the cavity and in communication with the cavity and the passageway,
the aspiration control vent including a linear slot located in the upper surface of the raised member,
the slot having a top portion and a bottom portion,
the slot extending parallel to the passageway,
the bottom portion of the slot having downwardly inclined bottom surfaces and a centrally located circular opening open to the top portion of the cavity,
the downwardly inclined bottom surfaces and the centrally located opening located below the upper surface of the raised member,
a removably attached specimen collecting vial having an open top in airtight attachment to a bottom portion of the cavity,
a separate removable flexible tube member extending from within the cavity and vial through the passageway and outwardly beyond the forward end for a predetermined distance,
the tube member including an airtight seal with the passageway,
a removable length of a metal hollow tube extending outwardly from the forward end and encircling the flexible tube in an airtight manner,
the rearward end including a connector connecting the passageway to a source of vacuum, and
the top portion of the slot adapted to be covered, partially covered and uncovered by moving the thumb of an operator on the upper surface of the raised member to incrementally adjust the vacuum pressure in the passageway.

2. The aspirator of claim 1 wherein:
the source of vacuum is operatively connected to a regulator to regulate the vacuum pressure in the tube member.

3. The aspirator of claim 1 wherein:
the top portion of the linear slot is adapted to be covered, partially covered and uncovered by moving the thumb of an operator on the upper surface of the raised member thereby incrementally adjusting the vacuum pressure in the tube member.

4-6. (canceled)

7. An aspirator for collecting fluids from remote human body cavities comprising:
a handle member having a top wall, a bottom wall, a forward end, a rearward end and a centrally located cavity opening through the bottom wall,
a longitudinal passageway extending through the handle member communicating the cavity with the forward end and the rearward end,
the top wall of the handle member having a centrally located cylindrical raised section enclosing a top portion of the cavity,
the raised section having a flat circular upper surface,
the upper surface of the raised section having a linear slot,
the slot having a top portion and a bottom portion,
the bottom portion of the slot having a centrally located circular opening open to the top portion of the cavity,
the slot extending substantially the diameter of the upper surface and parallel to the passageway,
the bottom portion of the slot and the centrally located opening being located below the upper surface of the raised section,
a specimen collecting vial having an open top removably attached to the bottom wall of the handle member below the cavity,
a flexible tube member extending from the cavity and vial through the passageway and outwardly from the forward end,
the tube member including an airtight seal with the passageway,
the rearward end having a connector connecting the passageway to a source of vacuum, an
a regulator member operatively connected to the source of vacuum, the regulator member operable to regulate the vacuum pressure in the tube member,
the top portion of the slot adapted to be covered, partially covered and uncovered by moving the thumb of an operator on the upper surface of the raised section thereby incrementally adjusting the vacuum pressure in the tube member.

8. The aspirator of claim 7 wherein:
the slot has downwardly inclined bottom surfaces terminating in the centrally located opening open to the cavity below the upper surface of the raised section.

9-10. (canceled)

11. A method of manually collecting samples of fluid from remote human body cavities comprising:
providing an elongated axially extending handle having a top surface, a bottom surface, a forward end, a rearward end and a centrally located cavity opening through the bottom surface,
gripping the handle between the cavity and the rear end,
providing a longitudinal passageway extending longitudinally through the handle from the forward end to the rearward end of the handle, the passageway communicating the cavity with the forward end and the rearward end,
providing a separate removable flexible tube member extending from within the cavity through the passageway and outwardly beyond the forward end of the handle,
aspirating a sample of fluid from a remote human body cavity through the tube member into the cavity with vacuum pressure,
incrementally adjusting the vacuum pressure in the tube member to control the aspiration of the sample of fluid from the body cavity,
providing the handle with a cylindrical raised member extending upwardly from the top surface of the handle and enclosing a top portion of the cavity, the raised member having a circular upper surface, the upper surface having a linear slot extending substantially the diameter of the upper surface and parallel to the passageway,
providing the linear slot with downwardly inclined bottom surfaces terminating in a centrally located circular opening open to the cavity,
recessing the downwardly inclined bottom surfaces and the centrally located opening below the upper surface of the raised member,
moving the thumb of an operator on the upper surface of the raised member to incrementally cover, partially cover and uncover the linear slot to variably regulate the vacuum pressure in the tube member,
collecting the sample of fluid in a sample vial having an open top in communication with the cavity,
removably attaching the sample vial to the handle in airtight attachment to the cavity, and
providing a regulator to regulate the vacuum pressure in the tube member.

12. The method of claim 11 wherein:
the body cavity is the middle ear.

13-16. (canceled)
